(19) United States
(12) Patent Application Publication
(10) Pub. No.: US 2007/0276352 A1
(43) Pub. Date: Nov. 29, 2007

(54) REMOVABLE DEVICE AND METHOD FOR TISSUE DISRUPTION

(75) Inventors: Michael D. Crocker, Half Moon Bay, CA (US); Steve Trebotich, Newark, CA (US); Daniel Kraft, Stanford, CA (US)

Correspondence Address: LEVINE BAGADE HAN LLP, 2483 EAST BAYSHORE ROAD, SUITE 100, PALO ALTO, CA, 94303, US

(73) Assignee: StemCor Systems, Inc., Menlo Park, CA (US)

(21) Appl. No.: 11/750,287

(22) Filed: May 17, 2007

Related U.S. Application Data
(63) Continuation-in-part of application No. 10/454,846, filed on Jun. 4, 2003.
(60) Provisional application No. 60/384,998, filed on Jun. 4, 2002.

(51) Int. Cl. A61M 31/00; A61M 1/00

(52) U.S. Cl. 604/500; 604/35

(57) ABSTRACT

Devices and methods for extraction of body tissue from an enclosed body cavity are disclosed. The devices can have one or more whisks extending from the distal end of flexible or rigid cannula. The devices can have aspiration and/or irrigation systems configured to provide aspiration pressure and/or irrigate with fluid at the distal end of the cannula. The cannula can be configured to rotate and/or oscillate. Methods for using the devices to disrupt the matrix of cancellous bone or bone marrow and extract in vivo cancellous bone or bone marrow from a subject are also disclosed.

### CROSS-REFERENCE TO RELATED APPLICATIONS

This is a continuation-in-part of U.S. patent application Ser. No. 10/454,846 filed Jun. 4, 2003, which claims priority to U.S. Provisional Patent Application Ser. No. 60/384,998 filed Jun. 4, 2002, each of which is incorporated herein by reference in its entirety.

### BACKGROUND OF THE INVENTION

i. Field of the Invention

The invention related to a device and method for extraction of tissue from an enclosed body cavity.

ii. State of the Related Art

Bone Marrow is a rich source of pluripotent hematopoietic stem cells from which red blood cells, white blood cells, and platelets are formed. Bone marrow also contains additional populations of mesenchymal stem cells and other stem and progenitor cells which have the potential to repair and regenerate other tissues.

Since the early 1970's bone marrow and hematopoietic stem cell transplantation has been used to treat patients with a wide variety of disorders, including but not limited to cancer, genetic and autoimmune diseases. Currently over 60,000 transplants for a variety of indications are performed worldwide each year.

In autologous transplants, the patient has their own bone marrow collected prior to receiving high dose chemotherapy. Following high dose, myeloablative chemotherapy, which kills the majority of the patients' marrow stem cells, the stored autologous marrow or hematopoietic stem cells purified or enriched from the marrow are infused, and serves to improve the patient's hematolymphoid system.

In allogeneic transplants bone marrow, or other sources of hematopoietic stem cells derived from a full or partially human leukocyte antigen (HLA) matched sibling, parent or unrelated donor is infused into the recipient patient and following engraftment, serves to reconstitute the recipients hematopoietic system with cells derived from the donor.

Following myeloablative or non-myeloablative conditioning of a patient with chemotherapy and/or radiation therapy, the marrow is regenerated through the administration and engraftment of hematopoietic stem cells contained in the donor bone marrow.

In addition to hematopoietic stem cells and hematopoietic progenitors, bone marrow contains mesenchymal and other stem cell populations thought to have the ability to differentiate into muscle, myocardium, vasculature and neural tissues and possibly some organ tissues such as liver and pancreas. Research in preclinical animal studies and clinical trials suggest that bone marrow or some portion of the cells contained within marrow can regenerate tissues other than the hematopoietic system. This includes the ability for cells contained within the marrow to regenerate or facilitate repair of myocardial tissue following a myocardial infarction, and in the setting of congestive heart failure as evident by improved cardiac function and patient survival.

Bone marrow derived stem cells also show evidence for their ability to regenerate damaged liver and hepatic cells and portions of the nervous system including spinal cord. Additional organ systems including kidney and pancreas show benefit from bone marrow derived cells. Use of bone marrow and the stem cells contained within bone marrow may be of increasing clinical utility in the future treatment of patients. Furthermore a patient's own marrow has multiple applications in orthopedic procedures, including but not limited to spinal fusions, treatment of non-union fractures, osteonecrosis, and tissue engineering.

Stem cells utilized in transplantation are usually collected using one of two methods. In a first method known as a bone marrow harvest, bone marrow is directly accessed in and removed from the patient usually by multiple aspirations of marrow from the posterior ileac crest. The bone marrow harvest procedure is often performed in the operating room.

To perform a harvest of 500-1500 milliliters of marrow, multiple separate entries into the marrow cavity are required to in order to remove a sufficient amount of bone marrow. A bone marrow aspiration needle, such as a sharp metal trocar, is placed into the marrow space through the soft tissue and the outer cortex of the ileac crest. The aspiration needle enters less than 2 cm into the marrow cavity. Negative pressure is applied through the hollow harvest needle, usually by the operator pulling on an attached syringe into which 5-10 ml of marrow is aspirated. The needle and syringe are then removed.

After removing the collected marrow, the aspiration needle accesses a separate location on the ileac bone for another aspiration. This method of inserting the needle into the bone, removing the marrow, and removing the needle from the bone is performed on the order of 100-200 separate entries for an average patient to remove a volume of bone marrow required for transplantation.

Each puncture and entry into the marrow cavity accesses only a limited area of the marrow space, and the majority of practitioners only remove 5-10 milliliters of marrow with each marrow penetration. Pulling more marrow from a single marrow entry site otherwise results in a collected sample highly diluted by peripheral blood.

The bone marrow harvest procedure requires general anesthesia because the ileac crest is penetrated 100-300 times with a sharp bone marrow trocar. Local anesthesia is generally not possible given the large surface area and number of bone punctures required.

The donor needs time to recover from general anesthesia, and frequently suffers from days of sore throat, a result of the endotracheal intubation tube placed in the operating room.

Pre-operative preparation, the harvest procedure, recovery from anesthesia, and an overnight observation stay in the hospital following the procedure requires considerable time on behalf of the donor and the physician, and similarly additional expense. The cost of the procedure is often $10,000 to $15,000, which includes costs for operating room time, anesthesia supplies and professional fees, and post-operative care and recovery.

In addition to general operating room staff, the traditional bone marrow harvest procedure requires two transplant physicians. Each physician aspirates marrow from the left or right side of the ileac crest. The procedure itself usually takes approximately one and half hours for each operating physician.

Many donors experience significant pain at the site of the multiple bone punctures which persists for days to weeks.

Traditional bone marrow aspiration incurs a significant degree of contamination with peripheral blood. Peripheral blood contains high numbers of mature T-cells unlike pure bone marrow. T-cells contribute to the clinical phenomenon termed Graft vs. Host Disease (GVHD), in both acute and chronic forms following transplant in which donor T-cells present in the transplant graft react against the recipient (host) tissues. GVHD incurs a high degree of morbidity and mortality in allogeneic transplants recipients.

In a second method to collect stem cells for transplantation, mononuclear cells are removed from the donor's peripheral blood. The peripheral blood contains a fraction of hematopoietic stem cells as well as other populations of cells including high numbers of T-cells. In this procedure peripheral blood stem cells are collected by apheresis following donor treatment with either chemotherapy—usually cyclophosphamide—or with the cytokine Granulocyte Colony Stimulating Factor (GCSF). Treatment with cyclophosphamide or GCSF functions to mobilize and increase the numbers of hematopoietic stem cells circulating in the blood.

This collection method can be slow and time consuming. It requires the donor to first undergo five or more days of daily subcutaneous injections with high doses of the cytokine GCSF prior to the collection. These daily injections can be uncomfortable and painful and bone pain is a common side effect. Peripheral blood stem cells can not be obtained without this seven-plus day lead time.

Each day of apheresis costs approximately $3,000 including but not limited to the cost of the apheresis machine, nursing, disposable supplies and product processing. The patient often has to come back on multiple days in order to obtain an adequate number of stem cells. Costs for the GCSF drug alone approximate $6,000-$10,000 depending upon the weight of the patient.

Given the multiple days required to collect adequate numbers of hematopoietic stem cells, individual bags of peripheral blood product must processed and frozen separately. These bags are then thawed, and given back to the recipient patient at the time of transplant. The volume, and chemicals contained in the product freezing media can cause some complications, such as mild side effects, at the time of infusion.

Accordingly, there is a need for a minimally invasive, less expensive, time-efficient bone marrow harvest procedure with minimal complications which does not require general anesthesia, offers fast recovery time, and does not cause significant pain to the bone marrow donor.

### SUMMARY OF THE INVENTION

Devices and methods for manipulation and extraction of body tissue from an enclosed body cavity are disclosed. The device can have a hollow introduction or entry cannula that can have a trocar. The introduction cannula and a core element can penetrate body tissue, such as the marrow space contained within the ileac. A flexible aspiration cannula can then be inserted through the introduction cannula into body tissue and can be advanced through the body cavity.

Within the aspiration cannula there may be a stylet (e.g., an aspiration stylet). The stylet can aid in advancing the cannula through the cavity. The stylet can be removed to facilitate extraction of body tissue through the aspiration cannula.

The aspiration cannula can have inlet openings near the distal tip through which tissue is aspirated. At the proximal end of the aspiration cannula a negative pressure (i.e., suction) source can provide controlled negative pressure, for example, to increase the aspiration of tissue through the aspiration cannula into a collection reservoir. The aspiration cannula can be withdrawn and positioned for multiple entries through the same tissue entry point, for example, following different paths through the tissue space for subsequent aspiration of more tissue.

A device or apparatus that can disrupt and aspirate bone marrow and/or other tissue rapidly and for large volumes of cancellous bone (i.e., marrow) from a target bone such as the ileac, femur, humerus, other bone, or combinations thereof is disclosed. The target bone can be in vivo or in vitro.

The apparatus can include a lumen adapted to receive an elongated aspiration cannula. Following entry through the bone wall, the aspiration cannula maybe controlled to move in a linear or non-linear fashion within the marrow cavity. The aspiration cannula, for example while moving non-linearly, can access a majority of the bone marrow space through a single point of entry. Suction may be optionally applied to the aspiration cannula while accessing the marrow space to increase the harvest of the bone marrow or other aspiratable substances. The apparatus can also optionally check for a threshold amount of aspiratable substance obtained. Additionally, a controller in the apparatus can adjust the aspiration cannula or signal for the operator to adjust the aspiration cannula to enable further harvesting from the same bone wall entry point, or in the alternate from an alternative bone wall entry point.

As the devices and methods can access large volumes of marrow with each catheter insertion, the devices and methods can be moved to directly contact more of the marrow space and aspirate a more concentrated, less diluted aspirant. The aspirated bone marrow can be more concentrated in stem cells, for example, because the device can penetrate the pelvic cavity more broadly and thus the extracted material can be less diluted with blood drawn into the void created by the extraction. The decreased numbers of contaminating T-cells can lead to less Graft vs. Host Disease (GVHD) in allogeneic bone marrow recipients. Less total volume of bone marrow can be removed (e.g., as it is more concentrated).

As mentioned, the harvest (i.e., aspiration, extraction) performed with the devices and methods disclosed herein can utilize one access point into the marrow cavity on one or both sides of the body to remove a minimal total volume of material that is highly concentrated. A marrow access site can be the anterior ileac crest access site which can be easy to locate and access on a broad array of patients (from thin to obese) and utilizing this access site can also reduce harvest time.

The method described herein can be performed by a single operator with no operating room time, reduced support personnel, no anesthesiologist, and can also be performed with no significant lead or preparative time. The method can be performed on, among others, critically ill subjects, or bone marrow donors who could not easily tolerate multiple surface puncture wounds for rapidly obtaining marrow and/or stem cells derived from marrow for use in immediate or long-term follow-on therapeutic interventions. Furthermore, the devices and methods disclosed herein can aspirate bone marrow, remove fat, aspirate blood and muscle, or combinations thereof, through a single skin (and bone, where applicable) puncture site into the tissue space (e.g., marrow cavity).

The device and method disclosed herein can also control the directionality of the cannula enough within the marrow cavity such that the device can access a majority of bone marrow space in a single bone or marrow cavity in vivo through a single point of entry. Alternatively, the device and method can access multiple diagnostic samples of bone marrow from disparate sites within a single marrow cavity. The device and method can also have aspiration suction controlled to aspirate bone marrow or fat, for example.

The device can have an elongated cannula having a flexible length, a hollow channel, a cannula first end and a cannula second end. The cannula first end can be open to provide fluid communication between the hollow channel and the outside of the cannula. Additionally, the device can include a motor which is rotatably connected to the cannula. The cannula may additionally include a tissue disruptor which is attached to or integral with the cannula, e.g., a whisk having a first end and a second end where the first end can be fixed to the cannula such that the whisk extends from the cannula. The second end can also be fixed to the cannula such that the whisk is configured in a semi-circular or closed loop configuration. The cannula can additionally include a second whisk extending from the cannula end.

The whisk can be configured to be rigid enough to substantially disrupt a first portion of a cancellous bone matrix when rotated by the motor to make the first portion removable from a surrounding portion of cancellous bone matrix while remaining flexible enough so as to inhibit or prevent its puncturing through cortical bone surrounding the body cavity.

To rotate the cannula and whisk, the motor can be configured to rotate the cannula at least at one operating speed from about 30 rpm to about 160 rpm. The device can include a mechanical transmission between the motor and the cannula to transmit the torque. A rotation-limiting resistor or slip-clutch in mechanical communication with the motor and the cannula may additionally be included to further control or limit the rotation of the cannula and whisk within the body space.

A pump in fluid communication with the hollow channel through the cannula may include an aspirant reservoir such that the hollow channel is in fluid communication with the reservoir. Additionally, an aspirant filter in fluid communication with the hollow channel and the aspirant reservoir may also be included to filter out undesirable material or debris. An irrigant reservoir holding a fluid, e.g., saline solution, may additionally be included for providing irrigation fluid which may be optionally perfused into the body space to facilitate tissue removal.

One method for removing cancellous bone or bone marrow from an in vivo bone in a subject may entail inserting the tissue disrupter into a first section of the cancellous bone or bone marrow into a patient by inserting a flexible hollow shaft into the first section of the cancellous bone. Prior to or upon insertion into the cancellous bone or bone marrow, the cannula and tissue disruptor may be rotated within the patient to disrupt the tissue matrix prior to or while optionally aspirating the disrupted portion of first section of cancellous bone or bone marrow into the cannula.

The method can also include re-positioning the flexible shaft into a second section of the cancellous bone and aspirating the disrupted tissue. The re-positioning of the flexible shaft into the second section of the cancellous bone can include completely or partially removing the tissue disrupter, e.g., a whisk-like disrupter, from the body cavity.

Additionally, the method can include irrigating the tissue matrix with a solution that can have saline, anesthetic, analgesic, anti-inflammatory, osteogenic powder or slurry, or combinations thereof. The method can further include filtering the aspirated disrupted portion.

### BRIEF DESCRIPTION OF THE DRAWINGS

FIG. 1 illustrates an exploded, partially schematic, view of a variation of the device for tissue disruption and aspiration.

FIG. 2 illustrates an assembled, partially schematic view of a variation of the device for tissue disruption and aspiration.

FIG. 3 illustrates an exploded, partially schematic, view of a variation of the device for tissue disruption and aspiration.

FIG. 4 illustrates an assembled, partially schematic view of a variation of the device for tissue disruption and aspiration.

FIG. 5 is a see-through view of a variation of the entry cannula with the core element.

FIG. 6 is a see-through view of a variation of the aspiration cannula.

FIG. 7 illustrates a variation of the aspiration cannula with one or more steering wires.

FIGS. 8a-8c illustrate variations of the perforated wall and cross-section of the aspiration cannula.

FIG. 9 illustrates a variation of the universal joint of the aspiration cannula.

FIG. 10 illustrates a variation of the squash plate of the aspiration cannula.

FIG. 11a and 11b illustrate variations of the preset degree of curvature of the aspiration cannula.

FIG. 12 illustrates a variation of the groove cup.

FIGS. 13-18 illustrate variations of the distal tip.

FIG. 19 illustrates a variation of inlet openings near the distal tip of the aspiration cannula.

FIGS. 20, 22, 25, and 27 are side views of variations of the distal tip of the aspiration cannula.

FIG. 21 is a front view of a variation of the distal tip of FIG. 20.

FIGS. 23 and 24 are front views of variations of the distal tip of FIG. 22.

FIG. 26 is a front view of a variation of the distal tip of FIG. 25.

FIG. 28 is a front view of a variation of the distal tip of FIG. 27.

FIG. 29 is a front view of a variation of the distal tip.

FIG. 30 illustrates a variation of the ports on the aspiration cannula.

FIG. 31 illustrates a variation of a method for using the tissue disruption and aspiration device.

FIG. 32 illustrates a variation of a method for entry on one side of the body with multiple aspiration paths.

FIG. 33 illustrates a variation of a method for harvesting bone marrow through one bone entry point.

FIG. 34 illustrates a variation of a method for harvesting bone marrow using several bone punctures and separate volume aspirations.

FIGS. 35-39 illustrate a variation of a method for using the tissue disruption and aspiration device.

FIG. 40 illustrates a variation of a method for rapid aspiration and collection of body tissue from within an enclosed body space.

FIG. 41 illustrates a variation of a method for entry on one side of the body with multiple aspiration paths.

### DETAILED DESCRIPTION OF THE INVENTION

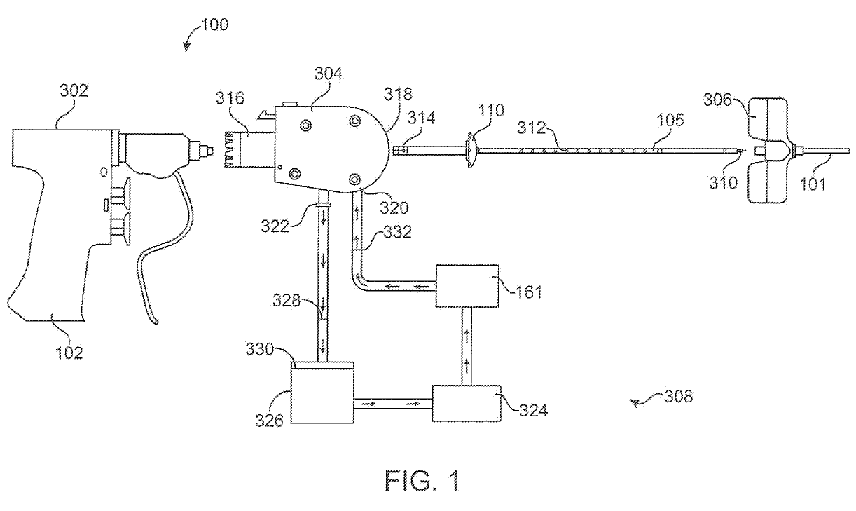

FIG. 1 illustrates a tissue disruption and aspiration device 100 that can aspirate and collect body tissue from within an enclosed body space in vivo or in vitro (also referred to as “aspiration device”). The aspiration device can have a drill 302, a connector and aspiration assembly 304, an aspiration cannula 105, an access trocar 306, and one or more fluid circuits 308.

The aspiration cannula 105 can attach to the connector 304 and/or drill 302 for ease of holding and operation such that the aspiration cannula 105 is in mechanical communication with the drill 302. The aspiration cannula 105 can be configured to be flexible or rigid and it may also include indentations, ridges, rings, visualization markers 312, or combinations thereof, for example to alter the flexibility of the aspiration cannula 105 along the entire length or a portion of the length of the aspiration cannula 105. The visualization markers 312 can be optionally radio-opaque and/or echogenic.

The aspiration cannula 105 may further include a rotational interface 314 configured to rotationally attach or couple to the connector 304 and/or the drill 302 for transmitting the rotational torque from the drill 302 to the cannula 105.

The aspiration cannula 105 can further include a guard and/or a squash plate 110 to prevent over-insertion of the aspiration cannula into the connector 304 and/or the drill 302. The guard can non-rotationally attach to the connector 304 and/or the drill 302 such that during use, the guard can remain rotationally constant. The guard may further cover a gap between the aspirant cannula 105 and the connector 304 and/or drill 302, for example, to prevent the operator from pinching his/her hands in the device 100 while the aspirant cannula 105 is rotating.

The aspirant cannula 105 can further include one or more control wires along the length of the aspirant cannula 105 (e.g., see FIG. 10). The squash plate 110 can be attached to the control wires such that the squash plate 110 can be manipulated by hand and/or by the connector 304 and/or by the drill 302 to steer, bend, flex, or combinations thereof, the distal end of the aspiration cannula 105.

The distal end of the aspiration cannula 105 can have a tissue disruptor, e.g., a whisk 310, which may be fixed, coupled, or otherwise integrated with the distal end of the aspiration cannula 105, as described in further detail below. The aspiration cannula 105 can facilitate aspiration and/or irrigation by defining one, two, or more lumens, for aspirating concurrently or subsequently to irrigating.

To provide an initial entry pathway into and through the cortical bone and into the medullary cavity, an access trocar 306 may be used which has an entry cannula 101 which defines an entry cannula channel that can pass through the length of the access trocar 306. The access trocar 306 can have one or more handles extending laterally and the entry cannula 101 can be configured to drive through cortical bone. Once the trocar 306 has been inserted and desirably positioned within the cortical bone creating an entry point, the aspiration cannula 105 may be passed through the entry cannula channel 101 and into the tissue matrix; accordingly, the channel 101 has a diameter which can reasonably accommodate the outer diameter of the aspiration cannula 105.

The connector and aspiration assembly 304 can have a drill interface 316 which mechanically couples the drill 302 and the connector 304 to one another via a removable interface which allows the drill interface 316 to couple and de-couple from the drill 302 itself. The connector and aspiration assembly 304 and/or the drill 302 can additionally include a mechanical transmission, for example, to increase and/or decrease the transmitted torque or speed from the drill 302 to the cannula 105. The connector and aspiration assembly 304 and/or the drill 302 can further include a governor, for example, to limit the rotational speed of the drill 302 transmitted to the aspiration cannula 105. Such a governor can be configured as a resistor, slip-clutch, etc., or combinations thereof. The maximum rotational speed of the aspiration cannula 105 can be from about 30 rpm to about 160 rpm, for example about 120 rpm.

The connector and aspiration assembly 304 can be further configured to direct and/or control aspiration and/or irrigation between the fluid circuit 308 and the first and/or second lumen of the aspiration cannula 105. The connector and aspiration assembly 304 can removably attach to the aspiration cannula 105 at a cannula port 318 and the connector and aspiration assembly 304 can further include an irrigation port 320 and/or aspiration port 322, each of which can be configured to be removably attached to fluid lines. The connector and aspiration assembly 304 can be configured to place the irrigation port 320 in fluid communication with a lumen in the aspiration cannula 105, for example a first lumen. The connector and aspiration assembly 304 can be further configured to place the aspiration port 322 in fluid communication with a lumen in the aspiration cannula 105, for example a second lumen, or the same lumen the irrigation port 320 is in fluid communication with.

The fluid circuit 308 can further include a pump 324 which is in fluid communication with an irrigant reservoir 161 and/or an aspirant reservoir 326. The irrigant reservoir 161 can have an irrigant, for example, saline solution. The pump 324 can deliver positive fluid pressure, as shown by arrows, to the irrigant reservoir 161 while also providing negative fluid pressure (i.e., suction), as shown by arrows, to the aspirant reservoir 326. The pump 324 can also be configured to reverse direction, i.e., providing negative pressure to the irrigant reservoir 161, and positive fluid pressure to the aspirant reservoir 326, for example, during cleaning to backwash the fluid system or to perfuse fluid into the tissue matrix to facilitate aspiration of the disrupted tissue. In this case, the irrigant perfusion rate can be, for example, from about 1 to 2 cc/min to about 30 cc/min.

An optional first aspiration filter 328 can be positioned in the flow between the aspiration port 322 and the aspirant reservoir 326 while an additional optional second aspiration filter 330 can be positioned in the aspirant reservoir 326, e.g., near the inlet port. An optional irrigation filter 332 can also be positioned between the irrigant reservoir 161 and the irrigation port 320. The first aspiration filter 328 and/or the second aspiration filter 330 can have pore sizes about 10 μm. While filters are shown positioned within the fluid lines or reservoirs, filters may alternatively be positioned within the cannula 105 itself, e.g., near or at the distal tip, for filtering out undesirable debris during aspiration such that the debris is prevented from passing through the cannula 105 and/or connector and aspiration assembly 304.

The drill 302, having a handle 102 and controls 103, can include any number of drills which are available for surgical purposes as interface 316 may be configured with a standard interface to couple and de-couple from any conventional drill interface. Examples of such drills 302 may include, for example, drills from DePuy Mitek, Inc. (Raynham, Mass.), Aesculap, Inc. (Center Valley, Pa.), Universal Driver or C.O.R.E. Micro Drill, Impaction Drill, Universal Series Drill (e.g., UHT Drill, U Drill), or Saber Drill commercially available from Stryker Corp. (Kalamazoo, Mich.), etc..

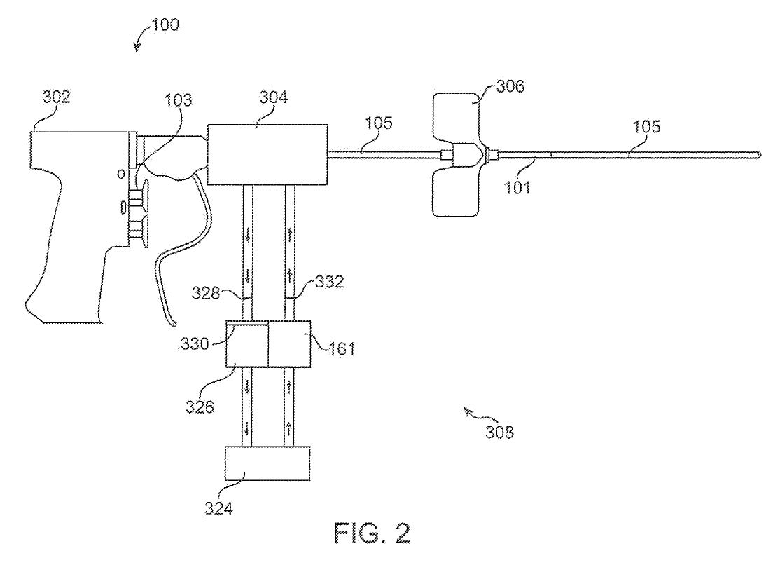

FIG. 2 illustrates another variation showing the aspirant reservoir 326 and the irrigant reservoir 161 integrated and/or attached to one another. As further shown, drill 302 is engaged to connector and aspiration assembly 304.

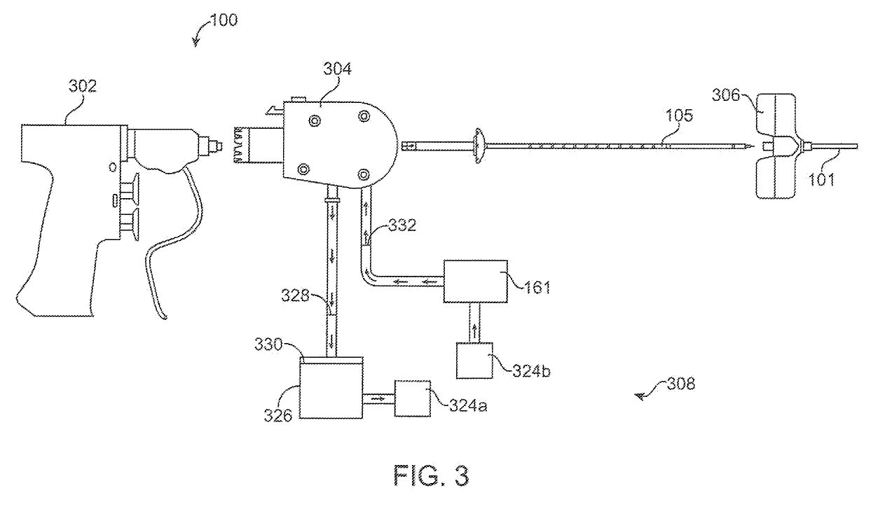

FIG. 3 illustrates another variation where the fluid circuit 308 can have separated irrigation and aspiration fluid flow sub-circuits. The irrigation sub-circuit can have an irrigation pump 324 while the aspiration sub-circuit can have an aspiration pump 324a separated from the irrigation pump 324b.

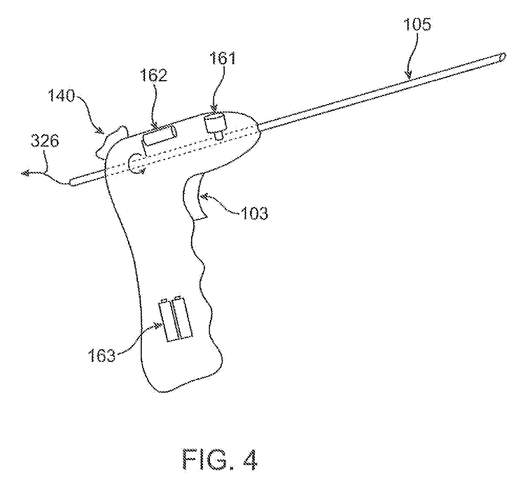

FIG. 4 illustrates an optional steering control 140 for steering, guiding, advancing, and/or retracting aspiration cannula 105 while aspiration cannula 105 in outside and/or inside bone marrow space (or other body tissue area). The aspiration cannula 105 can have one or more steering wires 107 (as shown in FIG. 10). The activation of the steering control 140 can contract or pull one or more of the steering wires 107. Contracting or pulling the steering wires 107 can result in the bending, curvature, and/or changing of direction of the aspiration cannula 105.

The steering control 140 can have a manual control, such as a handle, which can be moved to steer or manipulate the aspiration cannula 105. For example, forward movement of device 100 can advance the aspiration cannula 105 while backward movement of the device 100 can withdraw the aspiration cannula 105. Movement of the steering control 140 handle to different sides (e.g., to the left, right, up or down) curves or bends the aspiration cannula 105 to the corresponding side (e.g., to the left, right, up or down). The steering control 140 can have a powered control, such as a multi-way thumb-stick or one or more buttons for steering and/or advancing and retracting aspiration cannula 105 (shown in FIG. 4).

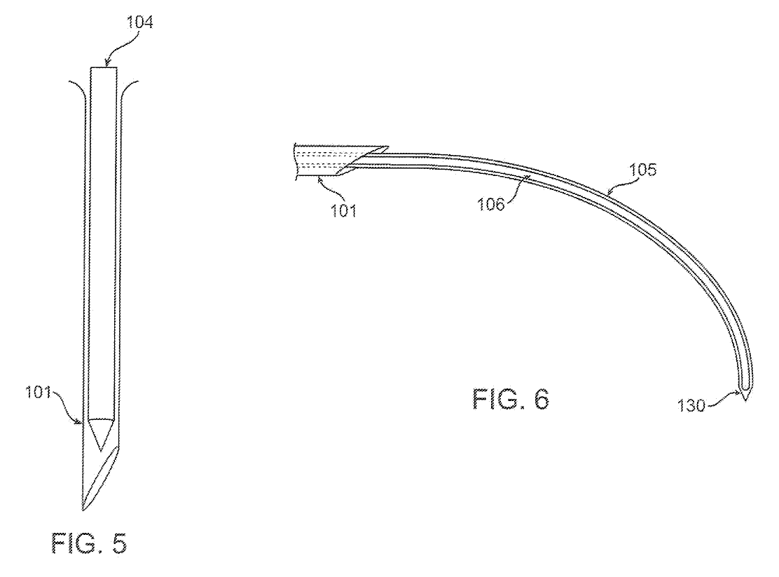

FIG. 5 illustrates entry cannula 101 with a core element 104. The entry cannula 101 comprises a needle with hollow central lumen accommodating a core element 104 for initial insertion into a bone marrow cavity or body tissue, for example through the anterior ileac crest, posterior ileac crest, lateral trocanter of the femur, or other location for example, for aspiration of bone marrow, fat, or other body tissue. The aspiration cannula 105 can enter the body tissue through the central lumen of the entry cannula 101, for example when the core element 104 is removed from the entry cannula 101.

The core element 104 comprises a rod, trocar or other element for breaking or piercing through the bone wall or other tissue boundary and creating an entryway for subsequent aspiration. The entry cannula 101 can be strong enough, or may not be strong enough, to break or pierce through the bone wall (e.g., cortical bone) without the help of core element 104.

An entry site in the bone wall can be created using a tool other than entry cannula 101 and/or core element 104, such as by a separate trocar or other sharp tool for breaking or piercing the bone wall. The aspiration cannula 105 can enter the bone through the break or piece in the bone wall (or other tissue area) for example, for the entry of aspiration cannula 105.

Once an entryway or entry site is created in the bone marrow and the entry cannula 101 can enter the bone marrow (or other body tissue intended for aspiration), the core element 104 can be removed leaving a hollow entryway or entry lumen with access to the medullary cavity.

FIG. 6 illustrates that the aspiration cannula 105 can translate through a hollow channel of the entry cannula 101. The aspiration cannula 105 can pass through the bone wall, or other tissue surface, and enter into the marrow or other tissue space. The aspiration cannula 105 can be flexible, for example flexing and curving to follow the bone marrow cavity or other tissue area. The aspiration cannula 105 can have a length of about 15 cm (6 in.) to about 41 cm (16 in.). The size of the aspiration cannula 105 can be selected for the size and anatomy of the patient and/or the bone marrow cavity or other body tissue area intended for harvest.

The aspiration cannula 105 optionally comprises a stylet 106 (e.g., an aspiration stylet). When inserted into the aspiration cannula 105, the aspiration stylet 106 can increase the structural strength of the aspiration cannula 105. The aspiration stylet 106 can transmit force to aid in advancing the aspiration cannula 105 through the marrow space or other tissue area. The marrow space can be the intramedullary bone marrow space of the ileac or femur bone. The aspiration stylet 106 can be straight or have a curvature prior to and following entry into body cavity through the entry cannula 101. The aspiration stylet 106 can be removed from the aspiration cannula 105 to allow aspiration of marrow (or other body tissue) through aspiration cannula 105. The aspiration stylet 106 can remove and/or disrupt tissue blockages within the aspiration cannula 105. The tissue blockages can be made from bone fragments, fat, coagulation, blood clots, other substances, or combinations thereof.

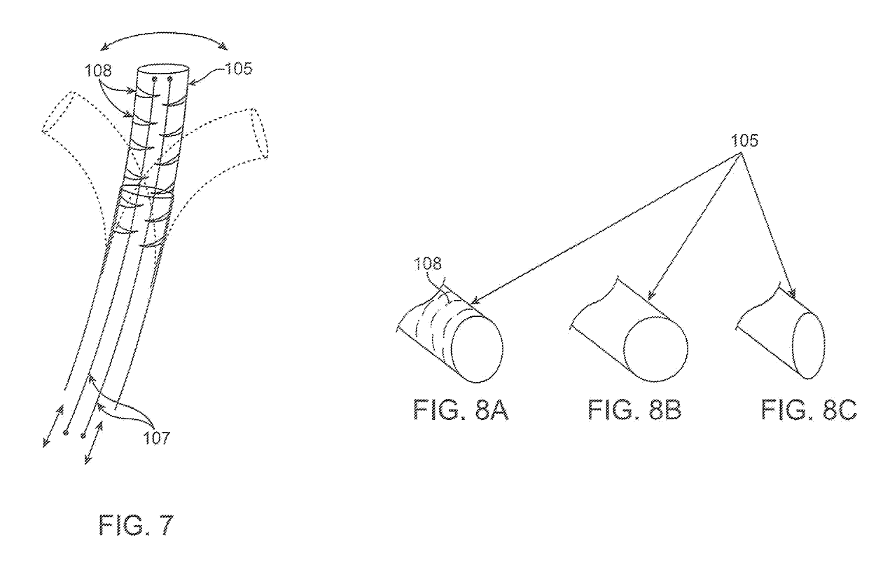

FIG. 7 illustrates that the aspiration cannula 105 can be steerable and directable. The aspiration cannula 105 can be equipped with one or more steering wires 107. Contraction or pulling of a steering wire 107 by an operator can flex (e.g., curve) the aspiration cannula 105 according to the direction and/or location of contracted or pulled steering wire 107.

FIG. 8 illustrates that the aspiration cannula 105 can be rigid or flexible. The aspiration cannula 105 can have grooves, slots or perforations 108 on the wall of aspiration cannula 105, as shown in FIG. 8a. The perforations 108 allowing for curvature and increased lateral flexibility, and/or oval cross-section for limiting axes of curvature. The aspiration cannula 105 can have or be made from material with shape-memory, for example a shape memory alloy (such as Nitinol), a shape memory plastic, or other metallic or non-metallic material with shape-memory, for example resulting in a curved profile of aspiration cannula 105, for providing directionality to aspiration cannula 105 upon aspiration cannula's 105 entry into the body tissue or body cavity. Alternatively, the cross-sectional profile of the cannula 105 may be varied as well from a circular profile, as shown in FIG. 8b, to an elliptical profile, as shown in FIG. 8c, to alter the flexibility characteristics.

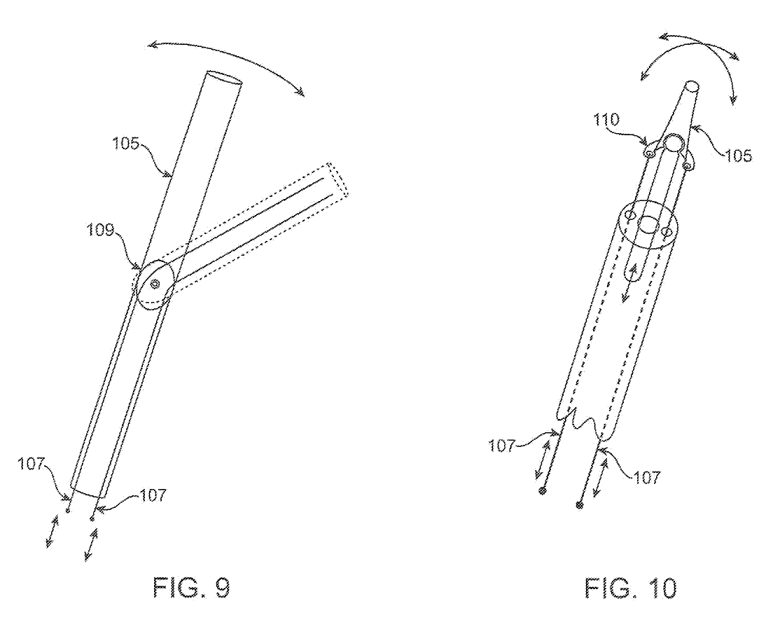

FIG. 9 illustrates that the aspiration cannula 105 can have a universal joint 109. The universal joint can be a pivot point. The universal joint can allow the contraction or pulling of steering wires 107 to result in steering and/or change of direction of aspiration cannula 105.

FIG. 10 illustrates that the aspiration cannula 105 can have a squash plate 110, as mentioned above, allowing the contraction or pulling of steering wires 107 to result in steering and/or change of direction of aspiration cannula 105.

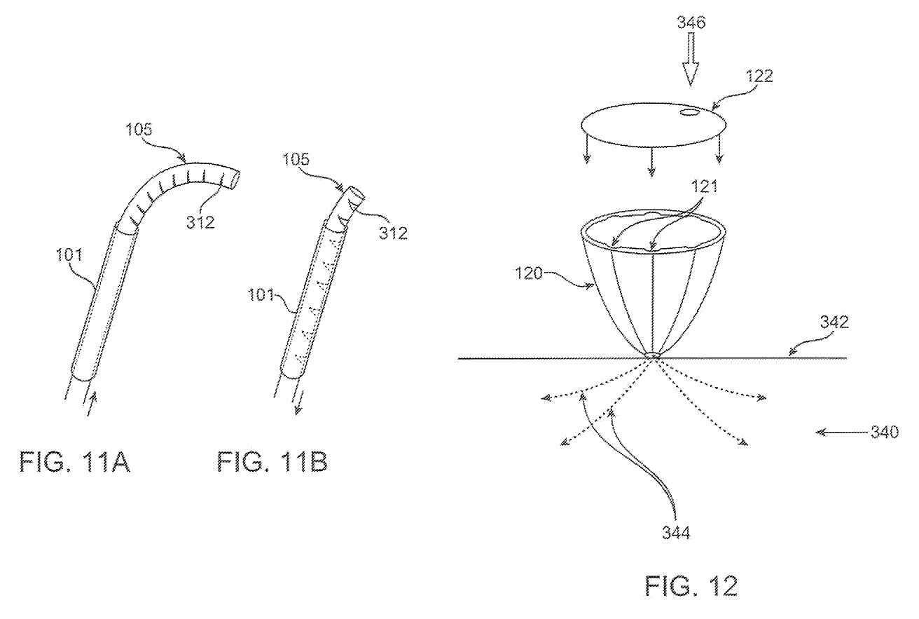

FIGS. 11a and 11b illustrate the aspiration cannula 105 in a configuration advanced out of the entry cannulas 101, as shown by arrow. The aspiration cannula 105 can have a preset degree of curvature such that after passing through the entry cannula 101 and into the bone cavity, the aspiration cannula 105 can assume a curvature according to the preset curvature, thereby assisting its direction when advancing within the cavity.

FIG. 12 illustrates that a groove cup 120 can guide the aspiration cannula 105, for example, through the bone surface 342 and into bone marrow 340 or other body tissue. The aspiration cannula 105 can be attached and/or slidably attach to an aspiration cannula entry 346 with a groove dial 122. Groove cup 120 comprises one or more grooves 121, a groove 121 providing directional entry of aspiration cannula 105 into bone marrow 340. The placement of the aspiration cannula 105 into an appropriate groove 121 allows entry of the aspiration cannula 105 into the bone marrow with directionality according to selected groove 121. The groove cup 120 can have a groove dial 122 for convenient selection of groove 121 and guiding of aspiration cannula 105 through selected groove 121 and into the bone marrow space. Various possible paths of the groove cup are shown by arrows, 344.

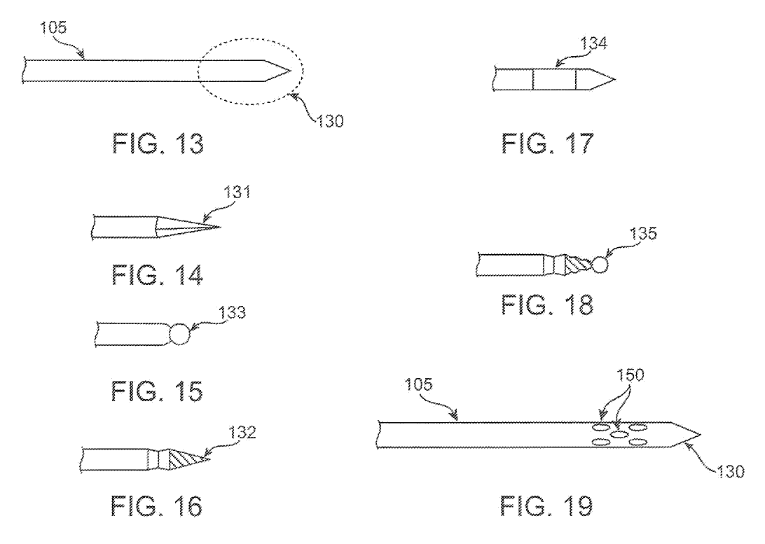

FIG. 13 illustrates that the device 100 can have a distal tip 130 at the distal end of aspiration cannula 105 or at the distal end of optional aspiration stylet 106, for advancing through the bone marrow cavity (or other body tissue).

FIG. 14 illustrates that the distal tip 130 can have a sharp tip 131. FIG. 15 illustrates that the distal tip 130 can have a transducer 133, such as a sonication device. The transducer can disrupt tissue, for example for penetrating and/or advancing through the cortical bone, cancellous bone (i.e., marrow) or other body tissue.

FIG. 16 illustrates that the distal tip can have a rotating drill tip 132. The rotating drill tip can be manual or motor-powered, for example powered by an electric motor 162 as shown in FIG. 4, with the motor 106 using power from batteries 163 or from an outside electrical source. The device 100 can have a variable speed controllable motor and/or reversible drill tip.

FIG. 17 illustrates that the distal tip 130 can have an ultrasound transducer or other navigation element 134 for providing navigation and/or visual guidance within bone marrow space (or other body tissue) to assist steering of aspiration cannula 105, such as providing feedback indicating proximity of distal tip 130 or aspiration cannula 105 to bone wall (or to other tissue boundary).

FIG. 18 illustrates that the distal tip 130 can be modified to have a rounded blunt tip 135. The distal tip 130 can be configured to not puncture out of the body space or cavity. For example the distal tip 130 can be dulled or softened to not pass through cortical bone during normal use. Upon encountering a wall or boundary (e.g., cortical bone) while the distal tip 130 is under pressure, the distal tip 130 can be configured to instead move sideway along a wall or boundary (e.g., cortical bone) upon encountering such a wall or boundary.

The device 100 can have radio-opaque and/or radio-transparent and/or echogenic markers or other materials. For example, the device 100 can be used with an imaging device, such as an X-ray or ultrasound device, for visual location of the aspiration cannula 105. The aspiration cannula 105 and/or other elements of the device 100 can be radio-transparent, and the aspiration cannula 1 05 can have a radio-opaque visual marker, such as a strip with visual distance markings showing how far aspiration cannula 105 has advanced into bone marrow space or other body tissue area, along the length of aspiration cannula 105.

FIG. 19 illustrates that the aspiration cannula 105 can have one or more inlet openings 150 near the distal tip 130. Marrow or other tissues can be aspirated by the application of negative pressure through the inlet openings 150. A negative pressure element, such as the aspiration pump, can be placed in fluid communication with the proximal end of aspiration cannula 105. The negative pressure element can apply a negative pressure resulting in aspiration (i.e., suction) of bone marrow or other body tissue into the aspirant reservoir. The negative pressure element can have a syringe. The negative pressure element can have a powered device. The negative pressure element powered device can be a wall-mounted continuous negative pressure device or other powered device for providing controlled negative pressure. The handle 102 can have a trigger element 103 (see FIGS. 1-4) that can control the aspiration negative pressure or degree of suction, for example by controlling a pressure gate for allowing a desired degree of negative pressure.

The aspiration device 100 can have a pain attenuating device for dampening pain and/or sensation during the aspiration procedure. The aspiration cannula 105 can have one or more elements for providing electrical nerve stimulation to the tissue harvest area. The electrical nerve stimulation can be configured to attenuate pain, for example, as shown in U.S. Pat. No. 6,159,163, Strauss et al, May/1998, which is incorporated herein in its entirety.

The inside wall of the entry cannula 101 and/or the aspiration cannula 105 can have an anticoagulant material such as heparin. The inside wall of the entry cannula 101 and/or the aspiration cannula 105 can be coated or otherwise lined. The anticoagulant can be configured to prevent blood and/or marrow from coagulating, for example to minimize hindering aspiration of marrow or body tissue. The entry cannula 101 and/or the aspiration cannula 105 can be flushed with anticoagulant solution to prevent and/or dissolve clots.

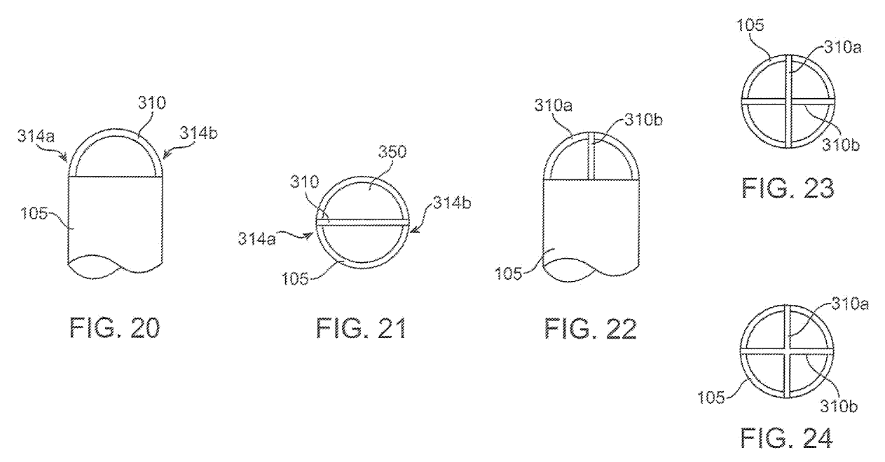

FIGS. 20 and 21 illustrate additional variations of the aspiration cannula 105 incorporating a tissue disruptor end effector configured in this variation as a whisk 310, as mentioned above. The whisk 310 can have a whisk first end 314a and a whisk second end 314b which can be attached to, or integral with, the distal end of the aspiration cannula 105. While the whisk 310 is illustrated as having a semi-circular or looped configuration, it may be configured in any number of shapes so long as clearance between the whisk 310 and cannula opening 350 is provided to allow for entry of the disrupted tissue therethrough. The whisk 310 can be resilient or deformable or alternatively flexible or rigid. The whisk 310 is also preferably rigid enough to disrupt cancellous bone yet flexible enough so as to not penetrate cortical bone during normal use.

FIG. 22 illustrates another variation with the cannula 105 utilizing two or more whisks 310a and 310b. The first and second ends of the whisks 310a, 310b can be attached to and/or integral with the distal end of the cannula 105. FIG. 23 illustrates an end view of a variation where that the first whisk 310a can be non-integral, unattached, or unconnected from the second whisk 310b while FIG. 24 illustrates likewise illustrates an end view of another variation where the first whisk 310a can be integral, coupled, or otherwise attached with the second whisk 310b.

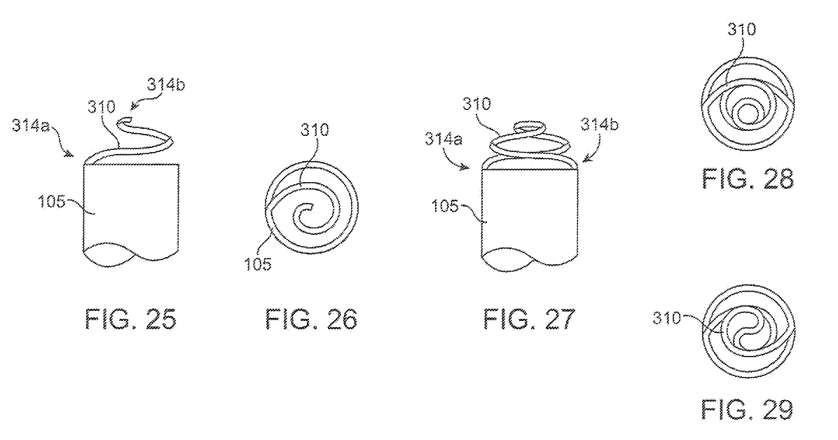

FIGS. 25 and 26 illustrate side and end views, respectively, of yet another variation where the whisk 310 can have a whisk second end 314b that is not attached to, or integral with, the aspiration cannula 105. Instead, the whisk 310 can have a helical or generally conical configuration where the second end 314b extends distally from cannula 105.

FIGS. 27 and 28 illustrate side and end views, respectively, of yet another variation in which a single whisk 310 can have a helical configuration where its first and second ends 314a and 314b can be integral with or attached to the distal end of the aspiration cannula 104. FIG. 29 illustrates a similar variation where the whisk 310 has a configuration similar to multiple oppositely-directed conical helixes.

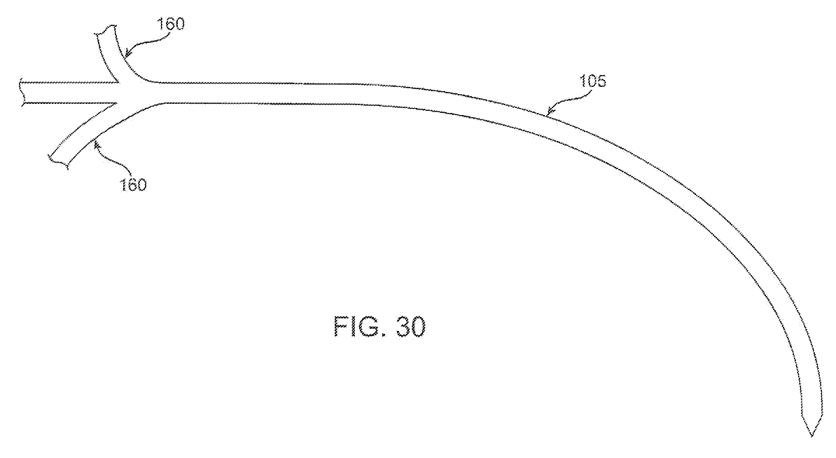

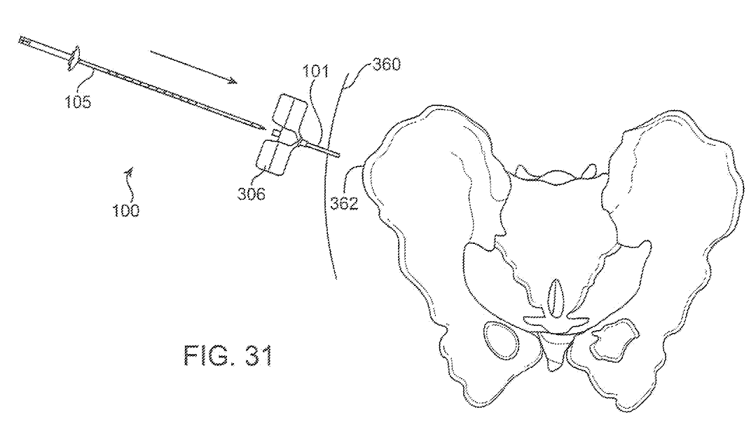

FIG. 30 illustrates that the aspiration cannula 105 can have one or more additional ports 160 through which material or liquid (such as the anticoagulant described above) can be administered. The ports in the aspiration cannula 105 can be ports through which a stylet can be passed into the aspiration cannula for unblocking or removing any blood or tissue clots which may occur. As shown in FIGS. 1 through 4, the aspiration device 100 can have an irrigant reservoir 161 for materials or liquids (such as anticoagulant described above) for administration, as shown in FIG. 9.

FIG. 31 illustrates a method of access where the access trocar 306 can be inserted percutaneously, as shown by the arrow, through the subject's skin 360 and into the target site, such as the ileac crest 362. With the trocar 306 desirable positioned through the ileac crest 362 and providing a entry port, the aspiration cannula 105 can then be introduced through the entry cannula 101, through the access trocar 362, and directly into the ileac crest 362.

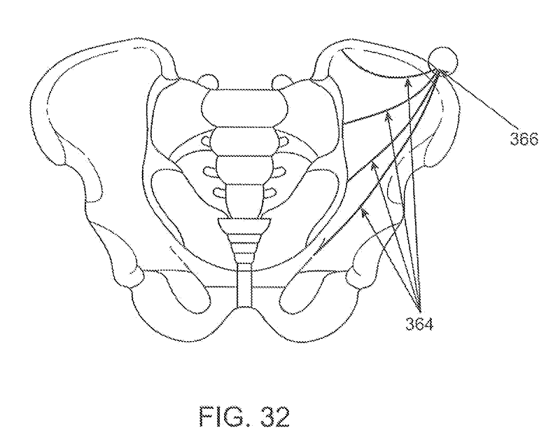

FIG. 32 illustrates that the length and/or diameter and/or flexibility and/or curvature of entry cannula 101 and/or aspiration cannula 105 can be chosen to accommodate different anatomies (e.g., different ages, bone sizes, amount of body fat, and other anatomical factors) and for the harvest of a range of body tissues, such as bone marrow, fat (e.g., liposuction), fluid in the abdomen of a patient (e.g., liver disease symptoms), or minimally invasive removal of a soft tissue mass such as a tumor. For example, a child may require a shorter, more flexible aspiration cannula 105. As another example, aspiration of bone through the lateral trocanter of the femur, or via the anterior ileac crest may require a shorter entry cannula 101 and/or aspiration cannula 1 05 than aspiration of bone marrow through the posterior ileac crest which may have more soft tissue above the bone. FIG. 32 shows various paths 364 that can be taken by the aspiration catheter 105 during the procedure through a single entry site 366 through the cortical bone.

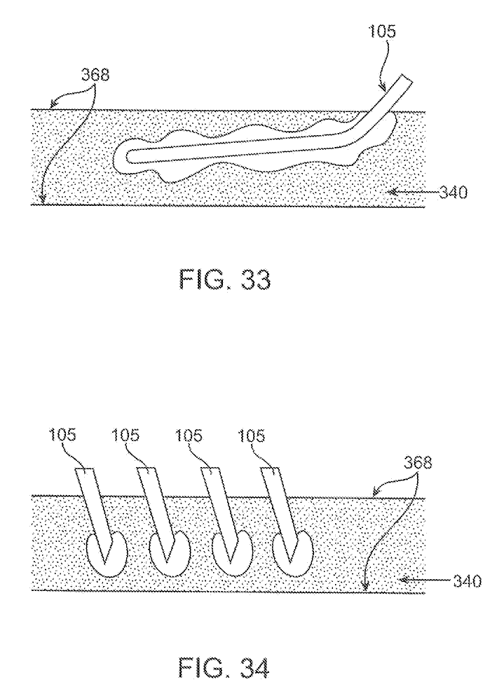

FIG. 33 illustrates that a single operator can harvest marrow 340, fat or other tissue through a single bone entry point in the cortical bone 368. FIG. 34 illustrates that one operator can harvest marrow, fat or other tissue through several dozen to hundreds of (bone) punctures and separate aspirations with one or more aspiration cannulas 105.

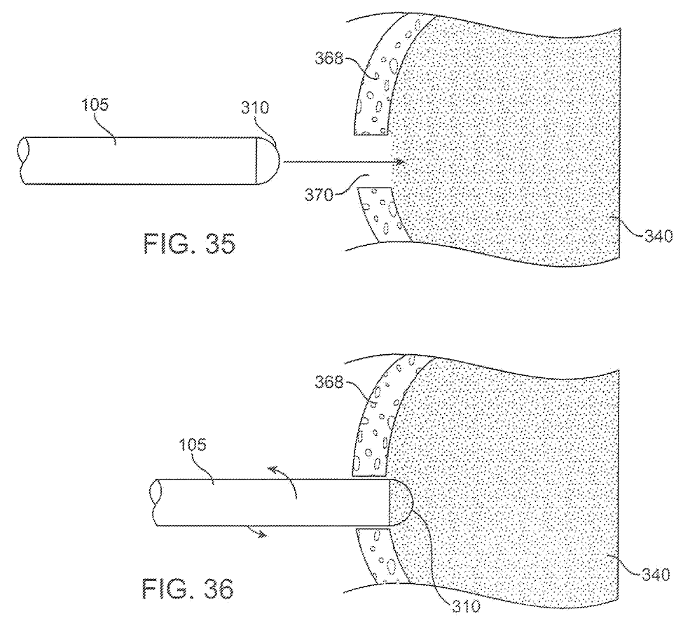

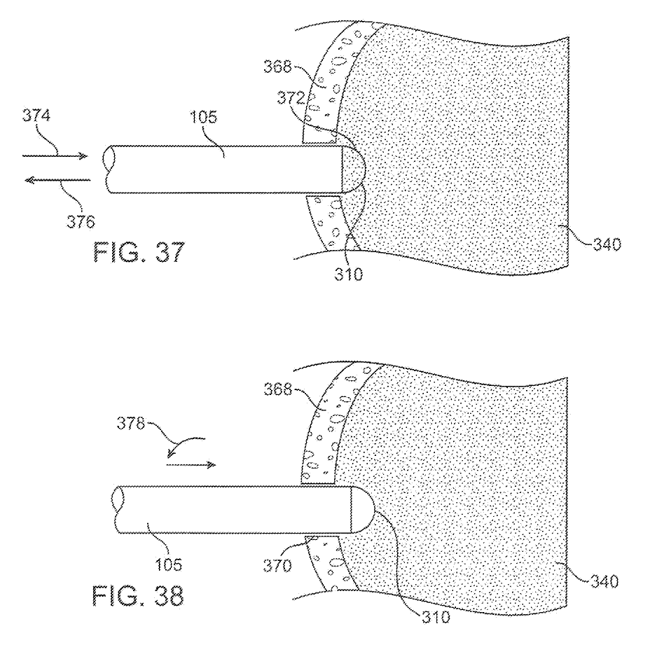

FIG. 35 illustrates a detail view illustrating the aspiration cannula 105 being translated, as shown by the arrow, through an entry port 370 cut into the cortical bone 368 by the access trocar 306 or another tool. FIG. 36 illustrates that once the whisk 310 is positioned wholly or at least partially within the cancellous bone 340, the aspiration cannula 105 can be rotated via the drill 302, as indicated by the arrows. As the matrix of the cancellous bone 340 around the whisk 310 is disrupted, as shown in disruption zone 372 illustrated in FIG. 37, the pump 324 can be activated (e.g., by a trigger or switch on the handle of the drill 302 or on the connector 304). The pump 324 can force, as shown by arrow 374, saline solution under pressure through one of the lumens 350 of the aspiration cannula 105. The irrigant can then mix under pressure with the disrupted cancellous bone. The pump 324 can produce suction, as shown by arrow 376, in one of the lumens 350 of the aspiration cannula 105 to remove the disrupted zone 372 of cancellous bone 340, as well as bone between the disrupted zone 372 and the lumen 350 through which the suction is delivered. The cannula 105 may be advanced distally along a first path into the cancellous bone 340 while rotating the cannula 105 and/or aspirating and/or perfusing.

FIG. 38 illustrates that the aspiration cannula 105 can be adjusted and repositioned, as shown by arrows 378. The adjustment and reposition can be concurrent with rotation of the aspiration cannula 105, for example to disrupt additional cancellous bone 340, or the adjustment and repositioning can occur without rotating the aspiration cannula 105. The aspiration cannula 105 can be repositioned through the same entry port 370 through the cortical bone 368.

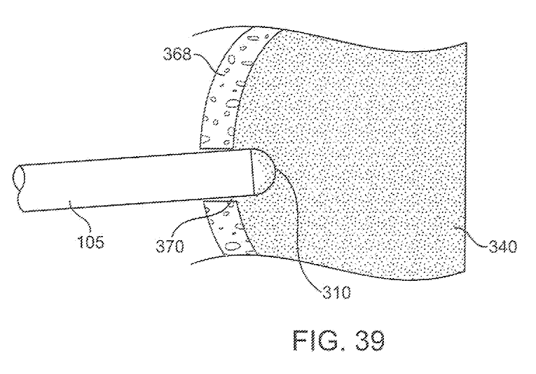

FIG. 39 illustrates that in the repositioned configuration, the whisk 310 can be surrounded by the cancellous bone 340 and the method shown in FIGS. 36 though 39 and described above can be repeated. The aspiration cannula 105 can be rotating throughout the method or the rotation can be stopped during repositioning.

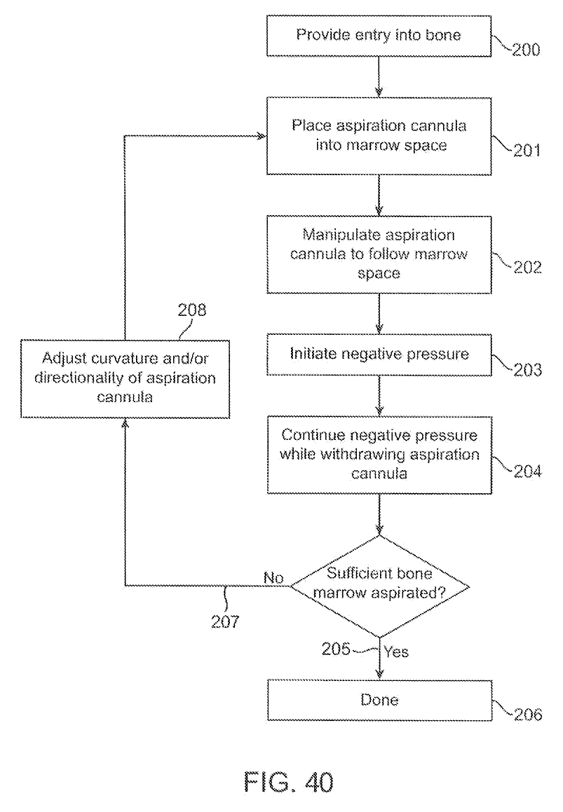

FIG. 40 illustrates a method for rapid aspiration and collection of body tissue from within an enclosed body space. After providing 200 an entry into the marrow using entry cannula 101 (and/or using core element 104, in which case the core element 104 of the entry cannula 101 is removed after providing the entry), ahollow entry lumen is left with access to the medullary cavity. Next, aspiration cannula 105 is placed 201 through the hollow entry cannula 101 and introduced into the marrow space. The aspiration cannula 105 is then manipulated 202 (using steering control 140) to move and follow the bone marrow cavity, assisted by the distal tip 130 the aspiration cannula.

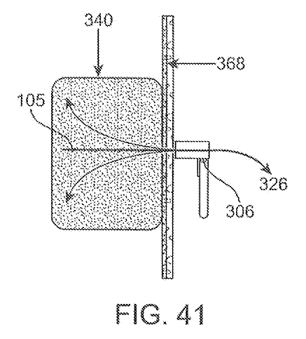

FIG. 41 illustrates that the aspiration cannula 105 will have a degree of flexibility and/or curvature allowing the aspiration cannula 105 to follow the cavity (e.g., the intramedullary bone marrow space of the ileac or femur bone). The aspiration cannula can have an ultrasound transducer device at the distal tip 130 of the aspiration cannula 105, for example to visualize the cavity (e.g., define the width of the cavity).

Once the aspiration catheter 105 is fully introduced into the body cavity, negative pressure can be initiated 203, using a syringe or a powered negative pressure device (e.g., the pump). As bone marrow is aspirated, the aspiration cannula 105 can be slowly withdrawn 204, with aspiration continuing as the aspiration cannula 105 is withdrawn. If 205 sufficient amount of bone marrow is aspirated 205, the aspiration process is complete 206. Otherwise 207, after withdrawal of aspiration cannula 105, the curvature and/or directionality of the aspiration cannula 105 can be adjusted 208, and the aspiration cannula 105 can be redirected through the entry into the bone marrow space and manipulated to follow a different path through the space and aspirating more bone marrow. This process can be repeated for example 3-4 times, resulting in its aspiration of bone marrow from the majority of the bore marrow space (for example the ileac crest). This process can be repeated on both sides of the body as needed (e.g., FIG. 32 shows an entry site on one side of the body with multiple aspiration paths).

Stem cells may be utilized to regenerate or improve function of damaged myocardium following a myocardial infarction, and may be useful in treating and preventing congestive heart failure. For example; a patient who has recently been diagnosed with a significant myocardial infarction and is brought to the catheterization suite, where interventional cardiologists perform angioplasty to open up a blocked coronary artery. Before, during or after the angioplasty procedure, a significant volume of bone marrow would be harvested. The bone marrow could be rapidly processed to enrich for hematopoietic stem cells or other populations or fraction of cells contained within bone marrow. These cells would then be delivered via catheter of other delivery device to the region of the heart which has undergone infarction and injury or death secondary to acute cardiac ischemia or other acute or chronic insults to the myocardial tissue. The delivered bone marrow or stem cell component contributes to regeneration of the myocardium or otherwise acts to improve cardiac function in the area of the infarct and leads to improved cardiac function and patient functional status and mortality. Optionally, marrow could be harvested separately from the initial cardiac catheterization procedure (for example 7 days after the MI, and in a separate procedure, stem cells or marrow enriched for stem cells could be delivered by any number of delivery mechanisms, for example by intracoronary or intramuscular injection. Use of a minimally invasive harvest device 100 would facilitate ease of harvest in patients who may be critically ill and not able to easily tolerate traditional marrow harvest procedures. In addition, minimally invasive harvesting of marrow has a role in intraoperative bone marrow harvesting for orthopedic applications.

As described above, there is the option of utilizing one or more aspiration cannulae 105 with preset or modifiable degrees of curvature and/or length and/or diameter and/or flexibility to adapt to different individual patients' anatomy and degree of ileac or other bone anatomy. Aspirated bone marrow can go directly into a bone marrow reservoir (e.g., the aspirant reservoir) or container through a closed system for initial storage and/or follow-on manipulation, such as filtering, stem cell enrichment, or other follow-on manipulation or treatment of bone marrow.

The apparatus and method shown herein provide many advantages for rapid aspiration and collection of body tissue from within an enclosed space. The directional control of the aspiration cannula by the operator enables the cannula to directly contact more of the marrow space and thereby aspirate a bone marrow that is more concentrated with stem cells than that available in the prior art. In addition, the harvest performed with the apparatus shown herein proceeds faster than prior art harvesting with a trocar since only one access point is required on each side of the body and less total volume of material is extracted. Finally, the procedure outlined above requires less time and reduced support personnel, thereby reducing costs for a procedure for harvesting bone marrow and/or tissue.

It is apparent to one skilled in the art that various changes and modifications can be made to this disclosure, and equivalents employed, without departing from the spirit and scope of the invention. Elements shown with any variation are exemplary for the specific variation and can be used on or in combination with any other variation within this disclosure.

We claim:

1. A device for tissue disruption comprising:
an elongated cannula having a flexible length and at least one lumen defined therethrough;
a tissue disruptor positioned upon a distal end of the cannula; and
an aspiration assembly having a first interface configured to rotatably connect to a proximal end of the cannula and a second interface configured to removably engage a motor.

2. The device of claim 1 wherein the second interface is configured to rotatingly couple to a drill.

3. The device of claim 1 wherein the cannula defines at least one additional lumen.

4. The device of claim 1 wherein the tissue disrupter comprises a whisk structure.

5. The device of claim 1 wherein the whisk defines a looped structure.

6. The device of claim 4 further comprising a second whisk structure positioned upon the distal end of the cannula.

7. The device of claim 1 wherein the tissue disrupter is attached to or integral with the cannula distal end.

8. The device of claim 1 wherein the tissue disruptor is configured to be rigid so as to disrupt a cancellous bone matrix.

9. The device of claim 8 wherein the tissue disruptor is further configured so as to inhibit puncture through cortical bone.

10. The device of claim 1 wherein the motor is configured to rotate the cannula from about 30 rpm to about 160 rpm.

11. The device of claim 1 further comprising a mechanical transmission engaged between the motor and the proximal end of the cannula.

12. The device of claim 1 further comprising a rotation-limiting resistor in electrical communication with the motor.

13. The device of claim 1 further comprising a slip-clutch in mechanical communication between the motor and the proximal end of the cannula.

14. The device of claim 1 further comprising a pump in fluid communication with the at least one lumen.

15. The device of claim 1 further comprising an aspirant reservoir in fluid communication with the at least one lumen.

16. The device of claim 15 further comprising an aspirant filter in fluid communication with the aspirant reservoir and at least one lumen.

17. The device of claim 15 further comprising an irrigant reservoir in fluid communication with the at least one lumen.

18. The device of claim 17 further comprising a saline solution contained within the irrigant reservoir.

19. A method for removing bone marrow from a subject, comprising:
removably coupling a motor to an interface of an aspiration assembly;
advancing a distal end of an elongated cannula having a flexible length into a body cavity of a patient, the cannula extending from the aspiration assembly;
disrupting a tissue matrix within the body cavity; and
aspirating the disrupted tissue matrix through at least one lumen defined through the cannula.

20. The method of claim 19 wherein removably coupling comprises rotatingly engaging the interface of the aspiration assembly to the motor.

21. The method of claim 19 wherein removably coupling comprises coupling a drill to the interface.

22. The method of claim 19 wherein advancing a distal end comprises introducing the cannula into a medullary cavity of the patient.

23. The method of claim 19 wherein advancing a distal end comprises introducing the distal end through a single opening defined along the body cavity.

24. The method of claim 19 wherein disrupting comprises rotating the cannula along a longitudinal axis via the motor.

25. The method of claim 19 wherein disrupting comprises disrupting cancellous bone within the body cavity.

26. The method of claim 19 wherein disrupting comprises rotating a whisk structure positioned at the distal end of the cannula.

27. The method of claim 19 further comprising irrigating the tissue matrix prior to aspirating.

28. The method of claim 27 wherein irrigating further comprises irrigating with saline.

29. The method of claim 19 wherein aspirating further comprises filtering the disrupted tissue matrix.

30. The method of claim 19 further comprising re-positioning the cannula into a second section of the tissue matrix.

31. The method of claim 30 wherein the re-positioning the cannula into the second section comprises partially removing the cannula from the body cavity.

32. The method of claim 19 wherein advancing comprises advancing the
